(19) United States
(12) Patent Application Publication
(10) Pub. No.: US 2021/0238386 A1
(43) Pub. Date: Aug. 5, 2021

(54) PRODUCE OF POLYVINYL ALCOHOL BIODEGRADABLE FILMS SOLUBLE IN WATER AT DIFFERENT TEMPERATURES (BIOLON)

(71) Applicant: BIO FUTURE WORLD LLC, Chestnut Hill, MA (US)

(72) Inventor: Ebrahim YAGHOUBI, Tabriz (IR)

(73) Assignee: BIO FUTURE WORLD LLC, Chestnut Hill, MA (US)

(21) Appl. No.: 16/972,533

(22) PCT Filed: Jun. 13, 2018
(86) PCT No.: PCT/IB2018/054290

§ 371 Date: Dec. 4, 2020

(51) Int. Cl. C08K 5/053; C08J 5/18; A61K 47/32

(52) U.S. Cl. CPC C08K 5/053; C08J 5/18; C08J 2300/16; C08J 2329/04; A61K 47/32

(57) ABSTRACT

In recent years humans have experienced many industrial and technological advances in various fields. One such technology that is widely used in nowadays is the plastic film technology. Plastic films of various thicknesses and sizes are widely used in different areas of life. But these products are very damaging to the environment and endanger the lives of humans All this however, no suitable product has yet been found to replace it. Therefore, we decided to produce a new film (biolon) to replace the old plastic films in order to overcome the problems related to the use of them. Biolon is biodegradable and soluble in different temperature water.

In this project is produce of polyvinyl alcohol biodegradable films soluble in different temperature water. This kind of PVA film called “Biolon”.

### TECHNICAL FIELD

This invention relates to a process for produce of different grade of Biolon from Polyvinyl alcohol polymer.

### BACKGROUND ART

Since the last few decades, films of different thicknesses are being used for different purposes. Earlier they were used for transferring, but now they are applied with various characteristics. Only one percent (1%) of the films are recoverable, convertible and recyclable. The main problems with these products are their non-biodegradability in the environment, environmental pollution and recycling.

In recent years, biodegradable films have found much importance in research. Various sources can be used as biodegradable films, for the production of biopolymers including polysaccharides, proteins and lipids. Being a polysaccharide, starch is of particular importance due to its low cost and abundance in nature. Various factors affect the properties of starch films, such as the source from which the starch is coming, as well as the ratio of ingredients starch is composed of. Starch films have advantages such as low thickness, flexibility and transparency, but it also has some disadvantages, including weak mechanical properties and its permeability to water vapor. Therefore, the use of starches alone for the production of films limits its application.

### SUMMARY OF INVENTION

In recent years humans have experienced many industrial and technological advances in various fields. One such technology that is widely used in nowadays is the plastic film technology. Plastic films of various thicknesses and sizes are widely used in different areas of life. But these products are very damaging to the environment and endanger the lives of humans. Most countries have plans not to use these kinds of films and they try to discourage people from using them, but they have not found a suitable product to replace it.

Therefore, we decided to produce a film (Biolon) to replace the popular films in order to overcome all these problems through the use of plastic films. Production of biodegradable films, which is soluble in water at different temperatures (Biolon), is a suitable substitute for all polymer films

### Technical Problem

In recent years humans have experienced many industrial and technological advances in various fields. One such technology that is widely used in today's machine life is the plastic film technology. Plastic films of various thicknesses and sizes are widely used in different areas of life. But these products are very damaging to the environment and endanger the lives of humans. Most countries have plans not to use these kinds of films and they try to discourage people from using them, but they have not found a suitable product to replace it. This type of film requires a period of around 300 to 400 years to decompose and return to the natural cycle. Therefore, we decided to produce a film (Biolon) to replace the popular films in order to overcome all these problems through the use of plastic films.

### Solution to Problem

Production of biodegradable films, which is soluble in water at different temperatures (Biolon), is a suitable substitute for all polymer films. They are environmentally friendly and have no toxic effects.

The main constituent ingredient of Biolon is polyvinyl alcohol polymer (PVA). Polyvinyl alcohol is a product of hydrolysis of polyvinyl acetate (PVAc), in which the hydrolysis reaction does not go to completion resulting in polymers with a certain degree of hydrolysis that depends on the extent of reaction. In principle, PVA is always a copolymer of PVA and PVAc. The degree of hydrolysis or the amount of acetate group in the polymer has an overall effect on the chemical properties, solubility and crystallizability of PVA. The degree of hydrolysis and polymerization affects the solubility of PVA in water. Research suggests that PVA with a high degree of hydrolysis has low solubility in water. Residual hydrophobic, acetate groups weaken intermolecular and intra-molecular hydrogen bonding of hydroxyl groups, in which case PVA and the resulting film can be soluble in water at temperatures above 70° C.

To produce low-temperature water-soluble film (Biolon), the water which is the best PVA solvent is chosen as the basic solvent 500 g of PVA 86 mole % hydrolyzed (purchased from Shandong Zhi Shang Chemical Co.) was added to 300 ml of water for 70° C. The obtained solution was stirred at 75° C. for 5 hours. After complete dissolution of PVA in water, to improve the physical properties of the final film, we add 400 g of glycerin 30%-sorbitol 30%-stearyl alcohol 40% composition as plasticizer to the reaction vessel and stir it for 2 hours at the same temperature. The obtained gel is spread over a flat surface and placed inside the oven at 180° C. for 3 minutes, and after passing through the cooling room, it is separated from the smooth surface and rolled.

The final formulation of the film along with viscosity control in the lab has had the best effect. The production of PVA packing film was done using this method.

To produce high-temperature water-soluble film (Biolon), first 500 g of hydrolyzed PVA 98% mole (purchased from Shandong Zhi Shang Chemical Co.) was added to 2 molar NaOH 80° C. The solution 2 molar NaOH 80° C. was used to eliminate Residual acetate groups. The obtained solution was stirred at 85° C. for 5 hours. After complete dissolution of PVA in water, to improve the physical properties of the final film, we add 350 g of glycerin 25%-sorbitol 30% stearyl alcohol 45% composition as plasticizer to the reaction vessel and stir it for 2 hours at the same temperature. The obtained gel is spread over a flat surface and kept inside the oven at 180° C. for 3 minutes, and after passing through the cooling room, it is separated from the smooth surface and rolled.

### Advantageous Effects of Invention

1. Degradable film in the environment without producing toxic materials

2. Soluble in water with different temperatures

3. Printable on it

4. Production in different sizes

5. Environment friendly

6. High mechanical and thermal resistance

7. Reasonable price

8. No need to bury to eliminate

9. No need for recycling

10. Non-use of hazardous chemical materials

11. Application in food packaging, sanitary, pharmaceutical, agriculture and etc.

### BRIEF DESCRIPTION OF DRAWINGS

This project and patent don't have drawing.

### DESCRIPTION OF EMBODIMENTS

Biolon is a biodegradable film and also, soluble in different temperature water.

### EXAMPLES

1. At first 410 g of warm water was added to 90 grams of PVA 86% hydrolysis degree. Afterward, added 10 g of 50% aqueous Sodium hydroxide solution as a catalyst. The mixture was stirred at 70° C. for 8 hours and further reacted for 20 hours at the room temperature. The resulting reaction mixture was neutralized and added dropwise to a large amount of methanol to reprecipitate the acetalized product of PVA formed. The acetalization degree of the product obtained was 5.2 mole %. A mixture of 100 parts by weight of the acetalized product with 9 parts by weight of sorbitol, 5 part by weight of stearyl alcohol and 3 parts by weight of glycerin was dissolved in water and cast to prepare a film having a thickness of 50μ. The dissolution time of the film obtained was about 40 seconds in water at 20° C.

2. At first 0.1% of nonionic anti-foam agent (Triton CF-32) and 9 ml of sodium hydroxide solution (50%) added to a 10% solution of polyvinyl alcohol 98% hydrolyzed and then solubilizing by heating the dispersion to a temperature of 81° C. to 105° C. with vigorous stirring. To eighty parts of the polyvinyl alcohol solution (10% solids), 1.25 parts of glycerin and 1.25 parts of sorbitol plasticizers is added. The casting solution is adjusted to 100 parts with water (9.5 parts water) to form an aqueous casting solution containing 12.5 parts (12.5%) of active ingredients, i.e., polymers and plasticizer. All ingredients are blended using vigorous agitation to assure homogeneity. From the aqueous casting solution films are prepared with both a 4-inch and 8-inch Gardener film casting knife using a blade clearance of 22-24 mils. The casting solution is deposited on either Mylar of plasticized polyvinyl chloride sheets as the casting surface. After drying in air overnight, clear films are obtained having a thickness of 38μ. The dissolution time of the film obtained was about 40 seconds in water at 60° C.

3. At first 13g of sodium hydroxide was added to 425 g of warm water. Afterward, added to 75 grams of PVA 98.5 mole % hydrolysis degree. The mixture was stirred at 70° C. Afterward, added 350 g of glycerin 25%-sorbitol 30% stearyl alcohol 45% composition as plasticizer to the reaction vessel and stirred it for 5 hours and further reacted for 20 hours at the room temperature. The resulting reaction mixture was neutralized and added dropwise to a large amount of acetone to reprecipitate the acetalized product of PVA formed. The acetalization degree of the product obtained was 7.7 mole %. The resultant acetalized product was dissolved in water, cast on a glass plate and dried at about 80° C. to prepare a film having a thickness of 30μ. The film was completely dissolved by dipping in water at 80° C. for about 45 seconds with stirring.

### INDUSTRIAL APPLICABILITY

The produced Biolon easily decomposes in nature or water. In order to attain an industrial base for sustainable development and conservation of natural habitats, the production of a new generation of polymeric materials is on the agenda of many advanced countries and assumes a special place. The use of Biolon in the packaging, pharmaceutical, agriculture and other industries has attracted the attention of manufacturers and consumers of the products of these industries.

### REFERENCE SIGNS LIST

1. Description

2. Technical Field

3. Background Art

4. Summary of Invention

5. Technical Problem

6. Solution to Problem

7. Advantageous Effects of Invention

8. Brief Description of Drawings

9. Description of Embodiments

10. Examples

11. Industrial Applicability

Reference to Deposited Biological Material

Sequence Listing Free Text

Citation List

Patent Literature

PTL1:

Non Patent Literature

NPL1:

1. Production of biodegradable film of polyvinyl alcohol, soluble in water at different temperatures with the following ingredients:
1. Water
2. Polyvinyl alcohol
3. Glycerin
4. Sorbitol
5. Stearyl alcohol

2. PVA film—Soluble in different temperature water—listed in claim No. 1, called “Biolon”.

3. Biolon film listed in claim No. 2 is intended for the production of all kinds of films in the pharmaceutical, food, industrial and sanitary grades.

4. Biolon film mentioned in claim No. 3 is intended to make nylon types in a variety of sizes and colors.

5. Biolon film mentioned in claim letter No. 4 is intended for the making of all types of packaging film and shopping bags.

6. The Biolon film mentioned in claim No. 5 has the ability to decompose in the environment in a period of 3 months.

7. Biolon film mentioned in claim No. 6 is capable of dissolving in water at different temperatures.

8. Biolon film mentioned in claim No. 7 does not release any toxic and harmful substances when it is decomposed.

9. The Biolon film mentioned in claim No. 8 is completely biodegradable.

10. The innovative element with this invention is at the speed of decomposition and harmlessness to the environment.
